(19) United States
(12) Patent Application Publication
(10) Pub. No.: US 2022/0101290 A1
(43) Pub. Date: Mar. 31, 2022

(54) PPE VERIFICATION SYSTEM AT POS

(71) Applicant: ZEBRA TECHNOLOGIES CORPORATION, Lincolnshire, IL (US)

(72) Inventors: Darran Michael Handshaw, Sound Beach, NY (US); Yuri Astvatsaturov, Lake Forest, IL (US); Edward Barkan, Miller Place, NY (US)

(21) Appl. No.: 17/036,310

(22) Filed: Sep. 29, 2020

(51) Int. Cl. G06Q 20/18; G06Q 10/06; G06Q 20/20; G16H 40/67; G16H 50/30; G06K 9/00; G08B 5/22; G06K 9/20

(52) U.S. Cl. CPC G06Q 20/18; G06Q 10/06311; G06Q 10/06315; G06Q 20/206; G06K 9/2018; G16H 50/30; G06K 9/00369; G08B 5/22; G16H 40/67

(57) ABSTRACT

Systems and methods for personal protective equipment (PPE) verification are disclosed herein. An example method for PPE verification includes capturing, by an image sensor associated with a checkout workstation, one or more images of an individual within a threshold proximity of the checkout workstation; analyzing, by one or more processors, the one or more images to determine whether the individual is wearing personal protective equipment (PPE); and triggering, by the one or more processors, one or more actions based on determining that the individual is not wearing PPE.

### BACKGROUND

The global Covid-19 pandemic has significantly changed the retail environment across the world. Stores now require customers to wear personal protective equipment (PPE) and practice social distancing, and must minimize the number of customers in the store at any time. This is a major change for retailers, and one that requires retailers to be vigilant of their customers' behavior in order to protect themselves and other customers from the spread of the virus. In particular, retailers must ensure that their stores are not contaminated, which could result in closing to disinfect, and may lead to revenue loss. Retailers must be particularly vigilant to avoid contamination at high-traffic locations in the store, such as checkout workstations, where customers may congregate in lines and may spread the virus by touching, coughing on, sneezing on, or otherwise breathing on other customers, store employees, or the checkout workstation itself, especially if customers are not wearing appropriate PPE or are not wearing their PPE properly.

### SUMMARY

In an embodiment, the present invention is a method, comprising: capturing, by an image sensor associated with a checkout workstation, one or more images of an individual within a threshold proximity of the checkout workstation; analyzing, by one or more processors, the one or more images to determine whether the individual is wearing personal protective equipment (PPE); and triggering, by the one or more processors, one or more actions based on determining that the individual is not wearing PPE.

In another embodiment, the present invention is a method, comprising: capturing, by an image sensor associated with a checkout workstation, one or more images of one or more first individuals and one or more second individuals; analyzing, by one or more processors, the one or more images to determine a distance between the one or more first individuals and the one or more second individuals; and triggering, by the one or more processors, one or more actions based on determining that the distance between any one of the first individuals and any one of the second individuals is less than a threshold distance.

In yet another embodiment, the present invention is a method, comprising: capturing, by an image sensor associated with a checkout workstation, one or more images of one or more first individuals and one or more second individuals; analyzing, by one or more processors, the one or more images to determine a distance between the one or more first individuals and the one or more second individuals; and triggering, by the one or more processors, one or more actions based on determining that the distance between any one of the first individuals and any one of the second individuals is less than a threshold distance.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 illustrates a block diagram of an example system including a logic circuit for implementing the example methods and/or operations described herein, including personal protective equipment (PPE) verification methods, social distancing verification methods, and/or disinfectant verification methods.

FIG. 2A illustrates an example of an individual properly wearing a face mask, while FIGS. 2B and 2C illustrate examples of users improperly wearing face masks.

FIG. 3A illustrates an example of properly worn gloves, while FIG. 3B illustrates an example of improperly worn gloves, and FIG. 3C illustrates an example of an individual's hands without gloves.

FIG. 4 illustrates a block diagram of an example process as may be implemented by the system of FIG. 1, for implementing example methods and/or operations described herein, including PPE verification methods.

FIG. 5 illustrates a block diagram of an example process as may be implemented by the system of FIG. 1, for implementing example methods and/or operations described herein, including social distancing verification methods.

FIG. 6 illustrates a block diagram of an example process as may be implemented by the system of FIG. 1, for implementing example methods and/or operations described herein, including disinfection verification methods.

FIG. 7 is a schematic, overhead view of an exemplary bi-optical checkout workstation at a retail checkout counter, for implementing the example methods and/or operations described herein.

FIG. 8 is a perspective view of the exemplary checkout workstation of FIG. 7 in isolation.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The present disclosure provides a personal protective equipment (PPE) verification system that can be implemented at a point of sale (POS), such as a checkout workstation. Specifically, the PPE verification system may utilize cameras or other image sensors positioned at the checkout workstation to capture images of individuals (e.g., customers or employees) as they approach the checkout workstation, and may analyze the images to determine whether an individual is wearing appropriate PPE for helping to prevent the spread of disease and/or whether the individual is wearing their PPE properly for preventing the spread of disease. Based on a determination that an individual is not wearing appropriate PPE and/or not wearing PPE properly, the PPE verification system may take a number of different actions to help prevent the spread of disease in various examples.

For instance, in some examples, the PPE verification system may trigger an automatic disinfection of the checkout workstation, e.g., after the individual finishes using the checkout workstation. Moreover, in some examples, the PPE verification system may prevent the individual from using the checkout workstation, or may close the checkout workstation to other individuals after the individual finishes using the checkout workstation (e.g., until the checkout workstation can be properly disinfected). Furthermore, in some examples, the PPE verification system may trigger an alert to a store employee indicating that the checkout workstation may be contaminated (e.g., so that the store employee can take steps to manually disinfect the checkout workstation after the individual finishes using the checkout workstation). Additionally, in some examples, the PPE verification system may trigger an alert to the individual who is not wearing appropriate PPE and/or not wearing their PPE properly, i.e., indicating that the individual should don his or her PPE, and/or providing instructions for correctly wearing the PPE. Moreover, in some examples, the PPE verification system may analyze the images of an individual who is not wearing PPE or not wearing PPE properly to identify the individual and send a notification to employees regarding the individual, or lock out the individual from using other checkout workstations, in the case of a repeat offender.

In some examples, the PPE verification system can also be configured to determine, based on the captured images, whether the individual is properly socially distanced from other individuals, and may generate alerts to be provided to the individuals indicating that the individuals should move further apart from one another, and/or generate alerts to be provided to store employees so that store employees may tell the individuals to move further apart from one another. Furthermore, in some examples, the PPE verification system can also be configured to determine, based on the captured images, whether the individual has applied disinfectant, and may prevent the individual from using the checkout workstation until he or she has applied disinfectant.

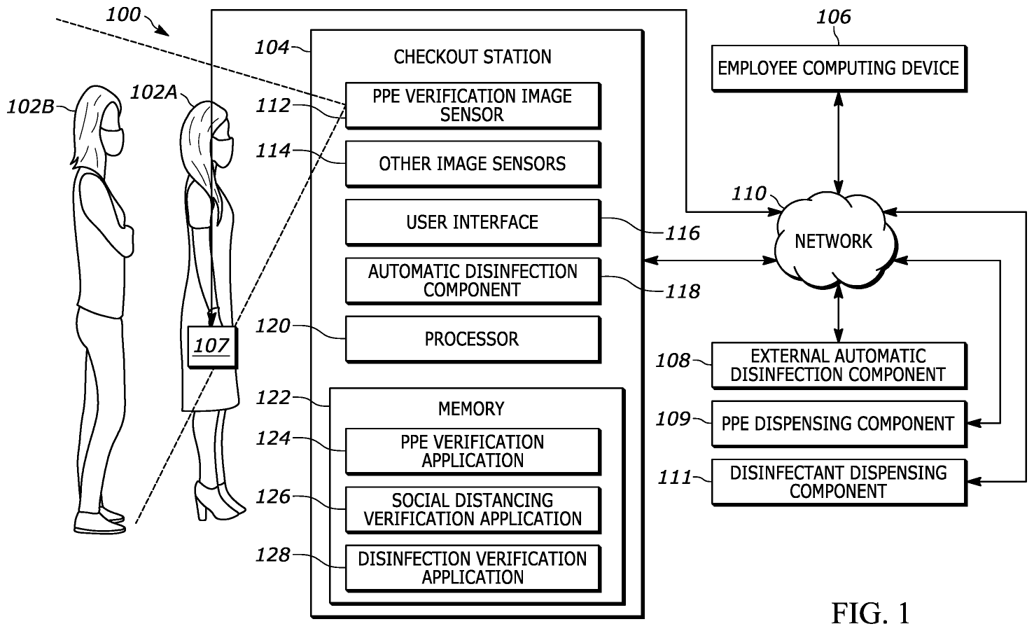

Referring now to the drawings, FIG. 1 illustrates a block diagram of an example PPE verification system 100. As shown in FIG. 1, one or more individuals 102A, 102B (e.g., customers, or clerks or otheremployees associated with the checkout workstation 104) may approach a checkout workstation 104 to complete a transaction. In some examples, the checkout workstation 104 may be configured to communicate with an employee computing device 106, a customer computing device 107, an external automatic disinfection component 108, a PPE dispensing component 109, and/or a disinfectant dispensing component 111, e.g., via a wired or wireless network 110.

The checkout workstation 104 may include one or more PPE verification image sensor(s) 112, as well as one or more other image sensor(s) 114, in some examples. As used herein, reference to an image sensor as a PPE verification image sensor 112 should not be read as limiting the image sensor to strictly PPE verification usage. The PPE verification image sensor 112 and the one or more other image sensors 114 may include two-dimensional cameras, depth cameras, infrared cameras, thermal cameras, or any other suitable image sensors in various examples. In some examples, the PPE verification image sensor 112 may be configured such that its field of view (FOV) includes faces, bodies, and/or hands of individuals who are currently using or are otherwise within a threshold proximity (e.g., six feet, ten feet, or another suitable threshold proximity) of the checkout workstation 104. For instance, in some examples, the PPE verification image sensor 112 may be angled or otherwise configured such that an individuals within images captured by the PPE verification image sensor 112 are generally within the threshold proximity of the checkout workstation 104.

The one or more other image sensors 114 may be configured such that their FOVs are directed to a product scanning region of the checkout workstation 104, e.g., so that the one or more other image sensors 114 may capture images of items to be purchased and/or barcodes associated with items to be purchased. Moreover, while in some examples, the PPE verification image sensor 112 and the other image sensors 114 may be different image sensors, in other examples a single image sensor or set of image sensors may be used for both PPE verification and product scanning purposes. Additionally, in some examples, the PPE verification image sensor 112 may be a component of the checkout workstation 104, in other examples, the PPE verification image sensor 112 may be separate from or otherwise external to the checkout workstation 104 (e.g., positioned above the checkout workstation 104 or a group of checkout workstations in a retail environment) and configured to communicate with the checkout workstation 104 via a wired or wireless network, such as the network 110.

Moreover, the checkout workstation 104 may include a user interface 116. The user interface 116 may be configured to display alerts, messages, notifications, and/or instructions to individuals who are using or are otherwise near the checkout workstation 104.

Additionally, in some examples, the checkout workstation 104 may include an automatic disinfection component 118. For instance, in some examples, the automatic disinfection component 118 may include an ultraviolet (UV) disinfecting light that activates to disinfect the checkout workstation 104. Moreover, in some examples, the automatic disinfection component 118 may include an aerosol spray washer that activates to spray disinfectant over affected areas of the checkout workstation 104 to disinfect the checkout workstation 104. For instance, the automatic disinfection component 118 may include a retractable spray nozzle that activates to spray disinfectant over at least a portion of the checkout workstation 104 and retracts when disinfection is complete. In some examples, the automatic disinfection component 118 may include an automatic wiper arm that activates to disinfect the checkout workstation 104 by wiping it down with disinfectant.

Furthermore, the checkout workstation 104 may include a processor 120 and a memory 122. The processor 120, which may be, for example, one or more microprocessors, controllers, and/or any suitable type of processors, may interact with the memory 122 accessible by the one or more processors 120 (e.g., via a memory controller) to obtain, for example, machine-readable instructions stored in the memory 122 corresponding to, for example, the operations represented by the flowcharts of this disclosure, including those of FIGS. 4, 5, and 6. In particular, the machine-readable instructions stored in the memory 122 may include instructions for executing a PPE verification application 124, a social distancing verification application 126, and/or a disinfection verification application 128. Moreover, the machine-readable instructions stored in the memory 122 may include instructions for reading barcodes in images captured by the image sensors 114, identifying items to be purchased depicted in images captured by the image sensors 114, and/or processing transactions for purchasing items. While the PPE verification application 124, social distancing application 126, and disinfection verification application 128 are shown as three separate applications in FIG. 1, the functionality described as being performed by each of these applications may be combined into one application or otherwise fewer total applications, or split into more total applications, and additional applications may be included in various embodiments.

Executing the PPE verification application 124 may include analyzing images of an individual 102A, 102B captured by the PPE verification image sensor 112 to determine whether the individual 102A, 102B is within a threshold proximity of the checkout workstation 104, and to determine whether the individual is wearing required PPE and/or whether the individual is wearing required PPE correctly. In some examples, the threshold proximity of the checkout workstation 104 may be a threshold proximity within which an individual 102A, 102B could spread disease via the checkout workstation 104 if not wearing PPE.

For instance, as discussed above, in some examples, the PPE verification image sensor 112 may be angled or otherwise configured such that an individuals within images captured by the PPE verification image sensor 112 are generally within the threshold proximity of the checkout workstation 104. For instance, the PPE verification image sensor 112 may be calibrated during installation such that its FOV corresponds with the threshold proximity of the checkout workstation 104. In other examples, the PPE verification application 124 may analyze the images of the individual 102A, 102B captured by the PPE verification image sensor 112 to determine whether the individual 102A, 102B is within the threshold proximity of the checkout workstation 104. For instance, the PPE verification application 124 may determine whether an individual 102A, 102B is within the threshold proximity of the checkout workstation 104 based on the apparent size of the individual 102A, 102B or features of the individual 102A, 103B (e.g., spacing between the eyes, head size, shoulder width, etc.) within the captured images and/or the apparent distance between the individual 102A, 102B and the checkout workstation within the captured images. That is, since the size of the PPE verification image sensor 112's FOV is known, the PPE verification application 124 may approximate the distance of the individuals 102A, 102B from the PPE verification image sensor 112.

Moreover, in some examples, when the PPE verification image sensor 112's FOV includes the floor and the feet of individuals 102A, 102B, the PPE verification application 124 may determine whether the individual 102A, 102B is within the threshold proximity of the checkout workstation 104 based on the location of the feet of the individuals 102A, 102B with respect to floor tiles (e.g., a threshold proximity may be five tiles from the checkout workstation 104), or with respect to markings placed on the floor. For instance, the PPE verification application 124 may determine that if an individual 102A is standing within a social-distancing-related marking six feet from the checkout workstation 104 indicating where the next individual in line to use the checkout workstation 104 should stand, the individual may be within the threshold proximity of the checkout workstation 104.

Additionally, in some examples, the checkout workstation 104 may include or be associated with a LIDAR or other time-of-flight (TOF) sensor, and the PPE verification application 124 may determine whether individuals 102A, 102B are within the threshold proximity of the checkout workstation 104 based on data captured by the LIDAR or other TOF sensor.

Required PPE may vary regionally (e.g., based on different country, state/province, or city requirements), and/or based on specific diseases that the PPE are intended to help prevent. The PPE verification application 124 may be updated (e.g., via the network 110) as PPE requirements change regionally or otherwise. As a few examples, PPE may include face masks (e.g., surgical face masks, cloth face masks, etc.), face shields, goggles, gloves, respirators, hairnets or other head coverings, and/or isolation gowns.

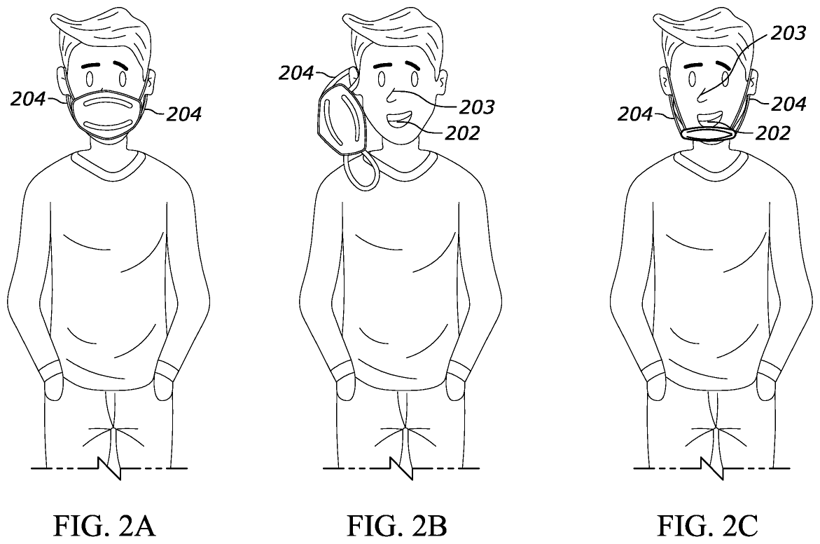

For instance, if the PPE verification application 124 is configured to determine whether an individual is wearing a face mask and/or whether the individual is wearing a face mask correctly (i.e., if required PPE includes a face mask), the PPE verification application 124 may analyze images of the individual's face to determine whether the individual's mouth and/or nose is visible, whether there is a demarcation or color transition below the eyes of the individual, indicative of a face covering, and/or whether strings or loops over the ears of the individual appear in the one or more images. For instance, as shown at FIG. 2A, when an individual is wearing his or her face mask correctly, his or her mouth 202 and nose 203 should not be visible, as they are blocked by the mask, and there should be a demarcation or color transition from the individual's skin color to the mask color under the individual's eyes. Furthermore, loops 204 or strings are visible over the individual's ears in FIG. 2A. In contrast, when an individual is wearing his or her face mask incorrectly, (e.g., the mask does not fully cover the nose and mouth) as shown at FIGS. 2B and 2C, the individual's mouth 202 and/or nose 203 may be visible, there may not be a demarcation or color transition from the individual's skin color to the mask color, and/or strings or loops 204 may not be visible over the individual's ears. Consequently, if required PPE at the checkout workstation 104 includes face masks, and the PPE verification application 124 determines that a mouth or nose appear in images of the face of an individual 102A, 102B within a threshold proximity of the checkout workstation 104, determines that there is no demarcation or color transition from the individual's skin color to the mask color in images of the face of the individual 102A, 102B, and/or determines that no loops 204 or strings appear over the individual's ears in the images of the face of the individual 102A, 102B, the PPE verification application 124 may determine that the individual 102A, 102B is not wearing PPE or is not wearing PPE correctly.

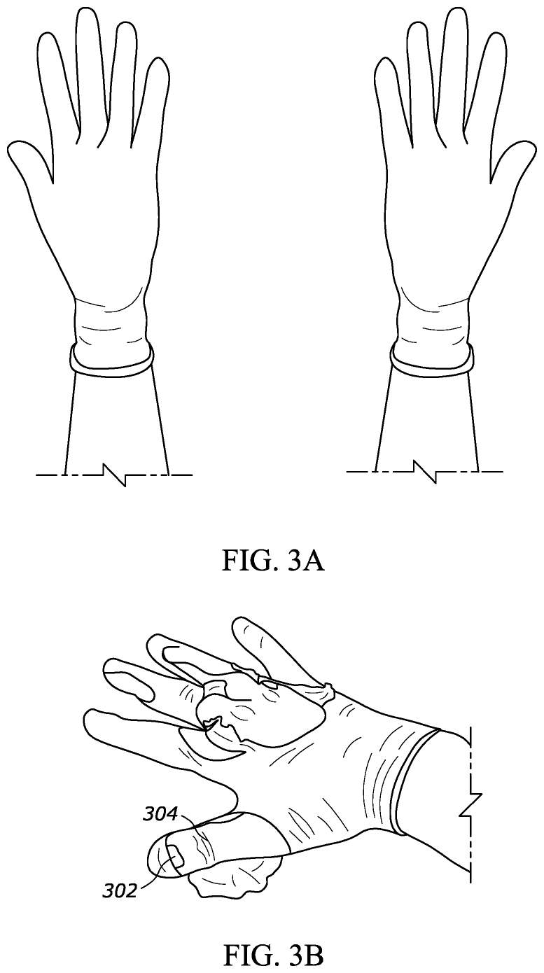

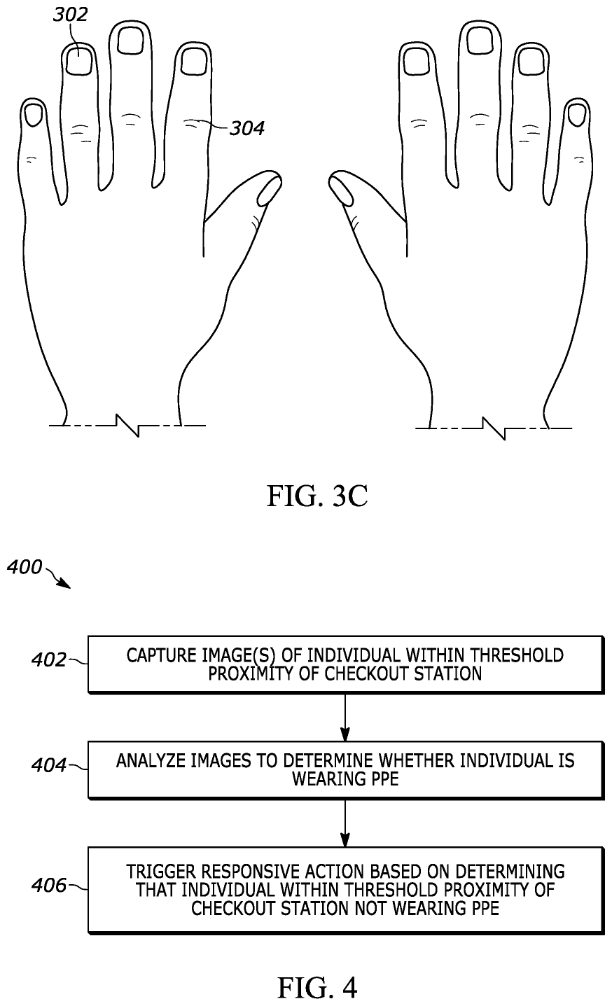

As another example, if the PPE verification application 124 is configured to determine whether an individual is wearing a pair of gloves, and/or whether the individual is wearing a pair of gloves correctly, the PPE verification application 124 may analyze images of the individual's hands to determine whether the individual's nails, cuticles, and/or hand wrinkles appear in the images. For instance, as shown in FIG. 3A, when an individual is wearing gloves properly, nails and/or cuticles 302, and hand wrinkles 304 should not be visible. In contrast, as shown in FIG. 3B, when an individual is wearing damaged gloves or is not wearing gloves properly, nails/cuticles 302, and/or hand wrinkles 304 may be visible. Similarly, as shown in FIG. 3C, when an individual is not wearing gloves at all, nails/cuticles 302, and/or hand wrinkles 304 may be visible as well. Consequently, if required PPE at the checkout workstation 104 includes gloves, and the PPE verification application 124 determines that nails, cuticles, and/or hand wrinkles appear in images of the hands of an individual 102A, 102B within a threshold proximity of the checkout workstation 104, the PPE verification application 124 may determine that the individual 102A, 102B is not wearing PPE or is not wearing PPE correctly.

In other examples, the PPE verification application 124 may perform a similar analysis for other types of PPE. For instance, to determine if an individual 102A, 102B is wearing goggles or a face shield, the PPE verification application 124 may analyze images of the face of the individual 102A, 102B to determine whether indications of glare appear over the face or eyes of the individual 102A, 102B. As another example, to determine if an individual 102A, 102B is wearing a hairnet or other head covering, the PPE verification application 124 may analyze images of the individual 102A, 102B to determine whether any hair appears in the images, or whether a demarcation line appears above the forehead of the individual 102A, 102B in the images, indicating a color transition between the skin tone of the individual 102A, 102B and a head covering.

Based on determining that the individual 102A, 102B is not wearing PPE, or is not correctly/properly wearing PPE, the PPE verification application 124 may trigger one or more responsive actions. In some examples, the PPE verification application 124 triggering the one or more responsive actions may be solely based on determining that the individual 102A, 102B is not wearing PPE, or that the individual 102A, 102B is not wearing PPE properly. In other examples, the PPE verification application 124 triggering the one or more responsive actions may be based on one or more additional factors. For instance, the PPE verification application 124 may trigger the one or more responsive actions based on a number of individuals 102A, 102B who have been within a threshold proximity of the checkout workstation 104 without wearing PPE or without wearing PPE properly since the checkout workstation 104 was last disinfected being greater than a threshold number of individuals (e.g., 2 individuals, 5 individuals, 10 individuals, etc.). As another example, the PPE verification application 124 may trigger the one or more responsive actions based on the total amount of time that various individuals 102A, 102B have been within a threshold proximity of the checkout workstation 104 without wearing PPE, or without wearing PPE properly, since the checkout workstation 104 was last disinfected being greater than a threshold amount of time (e.g., 15 total minutes, 30 total minutes, 100 total minutes, etc.). Additionally, in some examples, the PPE verification application 124 may trigger the one or more responsive actions based on determining that an individual within a threshold proximity of the checkout workstation who was not wearing PPE, or not properly wearing PPE, was exhibiting one or more signs of illness. For instance, in examples in which the PPE verification image sensor(s) 112 include a thermal camera, the PPE verification application 124 may trigger the one or more responsive actions based on the thermal camera detecting a high temperature (e.g., indicative of fever) associated with the individual 102A, 102B. As another example, the PPE verification application 124 may trigger the one or more responsive actions based on analyzing the images captured of the individual 102A, 102B to determine that the individual 102A, 102B is not wearing PPE, or is not wearing PPE properly, and appears to be coughing, sneezing, and/or speaking while within the threshold proximity of the checkout workstation 104 in one or more image.

In some examples, the PPE verification application 124's triggered responsive action may include activating the automatic disinfection component 118 of the checkout workstation 104 discussed above to automatically disinfect the checkout workstation 104 once the individual 102A, 102B has left the checkout workstation. The PPE verification application 124 may determine that the individual 102A, 102B has left the checkout workstation 104 based on, e.g., determining that the individual has completed a transaction at the checkout workstation or determining that no transaction activity has occurred at the checkout workstation for greater than a threshold period of time, or by analyzing images captured by the PPE verification image sensor 112 to determine that the individual 102A, 102B no longer appears within the FOV of the PPE verification image sensor 112, or to determine that the individual is greater than the threshold proximity from the checkout workstation 104.

Moreover, in some examples, the PPE verification application 124's triggered responsive action may include sending a signal to an external automatic disinfection component 108, which may be, for instance, a robotic cleaning apparatus configured to automatically travel to the checkout workstation 104 and disinfect the checkout workstation 104 once the individual 102A, 102B leaves the checkout workstation 104.

Additionally, in some examples, the PPE verification application 124's triggered responsive action may include automatically closing or locking the checkout workstation 104 to other individuals after the individual 102A, 102B leaves the checkout workstation 104 (e.g., until the checkout workstation 104 can be disinfected). Furthermore, in some examples, the PPE verification application 124's triggered responsive action may include closing or locking the checkout workstation 104 to the individual 102A, 102B, until the individual 102A, 102B properly dons PPE. For instance, an indicator light associated with the checkout workstation 104, or a feature of the user interface 116, may be activated or deactivated to indicate that the checkout workstation 104 is closed. Moreover, the checkout workstation 104 may be configured to cancel pending transactions, or refuse new transactions, while it is closed or locked. Additionally, in some examples, when the PPE verification application 124's triggered responsive action includes automatically closing or locking the checkout workstation 104, the PPE verification application 124's triggered responsive action may further include sending a notification, alert, or other message for presentation via the employee computing device 106 and/or the customer computing device 107 indicating that the particular checkout workstation 104 is closed. For instance, the notification may include an indication of an identifying number and/or store location associated with the closed checkout workstation 104.

Furthermore, in some examples, the PPE verification application 124's triggered responsive action may include analyzing images of individuals 102A, 102B who fail to don PPE or fail to properly don PPE to identify such individuals 102A, 102B, and automatically closing or locking the checkout workstation 104 (or other related checkout workstations) to the individual 102A, 102B if he or she attempts to use the checkout workstation 104 (or other related checkout workstations) without proper PPE at a later time.

In some examples, the PPE verification application 124's triggered responsive action may include generating an alert for the individual 102A, 102B and presenting the alert via the user interface 116 (e.g., audibly, visually, via a video, etc.). For instance, the alert may include a warning that the individual should don the required PPE, or instructions for how to properly/correctly don the required PPE. In some examples, the instructions may be general instructions for correctly wearing the required PPE, while in some examples, the PPE verification application 124 may analyze the captured images of the individual 102A, 102B to specifically determine ways in which the individual 102A, 102B is wearing the required PPE incorrectly, and the instructions may include instructions for how to correct the PPE based on the specific ways in which the individual 102A, 102B is wearing the required PPE incorrectly. As an example, if the individual 102A, 102B is wearing a mask, but the mask does not cover his or her nose, the instructions may indicate the individual 102A, 102B should move his or her mask such that it covers his or her nose. As another example, if the individual 102A, 102B is wearing a mask, but the mask is not tightly fitted to the face of the individual 102A, 102B, shadows may appear on the face of the individual 102A, 102B above or below the mask lines. Consequently, the instructions may indicate that the individual 102A, 102B should tighten his or her mask.

Additionally, in some examples, the PPE verification application 124's triggered responsive action may include generating an alert to be presented to an employee associated with the checkout workstation 104 via an employee computing device 106 that is separate from the checkout workstation 104. For instance, the alert to be presented to the employee may indicate that there is an individual 102A, 102B who is not properly wearing PPE at the checkout workstation 104. In some examples, the alert may include a captured image of the individual 102A, 102B, or other identification information associated with the individual 102A, 102B. Moreover, in some examples, the alert may indicate that the employee should instruct the individual 102A, 102B to don PPE, or should bring appropriate PPE to the individual. Additionally, in some examples, the alert may indicate that the employee should close the checkout workstation 104 until it can be disinfected, and/or that the employee should disinfect the checkout workstation 104 (e.g., once the individual 102A, 102B has left or otherwise finished using the checkout workstation 104).

Furthermore, in some examples, the PPE verification application 124's triggered responsive action may include automatically dispensing PPE (e.g., a mask, a face shield, goggles, gloves, a respirator, a hairnet or other head covering, and/or an isolation gown) for the individual, e.g., via a PPE dispensing component 109. In some examples, the PPE dispensing component 109 may be external to the checkout workstation 104, while in other examples, the PPE dispensing component 109 may be attached to, incorporated within, or otherwise associated with the checkout workstation 104. As another example, the PPE verification application 124's triggered responsive action may include automatically dispensing a stylus for the individual, via the PPE dispensing component 109, e.g., so that the individual can safely interact with the user interface 116 without touching it as needed.

Executing the social distancing verification application 126 may include analyzing images of one or more groups of one or more individuals (e.g., a first group of one or more individuals 102A and a second group of one or more individuals 102B) captured by the PPE verification image sensor 112 or the one or more other image sensors 114 to determine a distance between the first individual or group of individuals 102A and the second individual or group of individuals 102B. In some examples, the social distancing verification application 126 may determine the distance between the first individual or group of individuals 102A and second individual or group of individuals 102B in a similar manner as discussed above with respect to the PPE verification image sensor 114 determining the distance between an individual 102A, 102B and the checkout workstation 104 (i.e., determining whether the individual 102A, 102B is within the threshold proximity of the checkout workstation 104).

For example, the first group of individuals 102A may be a single individual 102A or a family of individuals 102A who do not need to socially distance between one another. However, social distancing may be required between the first individual 102A or first group of individuals 102A and a second individual 102B or second group of individuals 102B, as per local (country, state, city, etc.) requirements associated with the checkout workstation 104. In some examples, social distancing verification application 126 may determine whether an individual is part of a first group 102A or a second group 102B based on the individual's proximity to a first shopping cart or a second shopping cart (not shown) appearing in the captured images of the individuals 102A, 102B, or based on a number of total shopping carts appearing in the captured images of the individuals 102A, 102B. For instance, the first group of individuals 102A may be associated with a first shopping cart while the second group of individuals 102B may be associated with a second shopping cart.

In some examples, the social distancing verification application 126 may determine the distance between the first individual or group of individuals 102A and the second individual or group of individuals 102B based on analyzing the images of the individuals 102A, 102B to identify faces of the first individuals 102A and second individuals 102B, determining apparent sizes of each of the first individuals 102A and the second individuals 102B as the faces appear in the one or more images, and comparing the apparent sizes of the faces each of the first individuals 102A to the apparent sizes of the faces each of the second individuals 102B. That is, the faces of individuals closer to the PPE verification image sensor 112 and/or image sensor 114 will have larger apparent sizes than the faces of individuals further from the PPE verification image sensor 112 and/or image sensor 114, and differences in the apparent sizes of the faces of the first individuals 102A and second individuals 102B may indicate the relative distance of each individual or group of individuals from the image sensor 112, 114, and may thus indicate relative distances of the first individuals 102A and second individuals 102B from one another (especially if the individuals are in a line).

Additionally, in some examples, the social distancing verification application 126 may determine the distance between the first individual or group of individuals 102A and the second individual or group of individuals 102B based on analyzing the images of the individuals 102A, 102B to identify faces of the first individuals 102A and second individuals 102B, and may utilize a depth camera (e.g., the PPE verification image sensor 112 or one of the other image sensors 114) to determine depths associated with the faces of the first individuals 102A and second individuals 102B, which may indicate each of the individuals' distance from the depth camera and thus may indicate relative distances of the first individuals 102A and second individuals 102B from one another (especially if the individuals are in a line).

In any case, the social distancing verification application 126 may trigger one or more responsive actions based on determining that the distance between any one of the first individuals 102A and any one of the second individuals 102B is less than a threshold distance (e.g., six feet, ten feet, etc.) required based on social distancing rules. For instance, the social distancing verification application 126's triggered responsive action may include generating an alert to be presented to the individuals 102A, 102B (e.g., via the user interface 116 of the checkout workstation 104) indicating that the distance between any one of the first individuals and any one of the second individuals should be increased to greater than the threshold distance. As another example, the social distancing verification application 126's triggered responsive action may include generating an alert to be presented to an employee associated with the checkout workstation 104 (e.g., via an employee computing device 106) indicating that the distance between any one of the first individuals and any one of the second individuals should be increased to greater than the threshold distance, i.e., so that the employee may alert the individuals 102A, 102B that they should increase the distance between respective groups.

Executing the disinfection verification application 128 may include determining that an individual 102A, 102B is within a threshold proximity of the checkout workstation 104. In some examples, the disinfection verification application 128 may determine that the individual 102A, 102B is within the threshold proximity of the checkout workstation 104 based on analyzing images captured by the PPE verification image sensor 112 or other image sensors 114. Additionally, in some examples, the disinfection verification application 128 may determine that the individual 102A, 102B is within the threshold proximity of the checkout workstation 104 based on, e.g., the individual attempting to initiate a transaction (e.g., attempting to purchase an item or scan a barcode).

Further, the disinfection verification application 128 may determine an indication that the individual 102A, 102B has applied disinfectant to his or her hands, and initiate a transaction at the checkout workstation 104 (based on a request from the individual to initiate a transaction) responsive to determining that the individual 102A, 102B has applied disinfectant while in proximity of the checkout workstation 104. That is, in some examples, the disinfection verification application 128 may close the checkout workstation 104 to an individual 102A, 102B attempting to initiate a transaction until the disinfection verification application 128 determines that the individual 102A, 102B has applied disinfectant. In some examples, the disinfection verification application 128 may generate an alert for the individual (e.g., to be provided via the user interface 116) indicating that the individual must apply disinfectant before a transaction may be initiated.

In some examples, the disinfection verification application 128 may determine that the individual 102A, 102B has applied disinfectant based on analyzing images of the hands of the individual 102A, 102B captured by the PPE verification image sensor 112 or other image sensors 114 to determine whether the individual is applying disinfectant in the one or more of the images. For instance, the disinfection verification application 128 may analyze the images of the hands of the individual 102A, 102B to determine whether the individual 102A, 102B is activating a dispenser of disinfectant (e.g., the disinfectant dispensing component 111) in the one or more images. In some examples, the disinfectant dispensing component 111 may be external to the checkout workstation 104, while in other examples, the PPE dispensing component 109 may be attached to or incorporated within the checkout workstation 104. Moreover, in some examples, in some examples, the disinfection verification application 128 may determine that the individual 102A, 102B has applied disinfectant based on receiving an indication that a the disinfectant dispensing component 111 associated with the checkout workstation 104 has been activated. For instance, if the disinfectant dispensing component 111 is an automatic dispenser that includes a motion detector and dispenses disinfectant based on motion of hands beneath the dispenser, the disinfectant dispensing component 111 may send a signal to the checkout workstation 104 each time disinfectant is dispensed. As another example, if the disinfectant dispensing component 111 is a manual dispenser, the disinfectant dispensing component 111 may include a touch or pressure based sensor that indicates that a pump of the disinfectant dispensing component 111 has been pressed to pump disinfectant, and may send a signal to the checkout workstation 104 each time disinfectant is pumped. The disinfection verification application 128 may then determine if disinfectant has been dispensed and/or pumped at a time during which the images show the individual 102A, 102B within the threshold proximity of the checkout workstation 104.

FIG. 4 illustrates a block diagram of an example process 400 as may be implemented by the system of FIG. 1, for implementing example methods and/or operations described herein, e.g., including PPE verification methods as discussed as being performed by the PPE verification application 124 of the checkout workstation 104. At block 402, images of an individual within a threshold proximity of a checkout workstation may be captured by an image sensor. At block 404, the images may be analyzed to determine whether the individual within the threshold proximity of the checkout workstation appears to be wearing PPE. At block 406, a responsive action may be triggered based on a determination that the individual within the threshold proximity of the checkout workstation is not wearing PPE or is wearing PPE incorrectly.

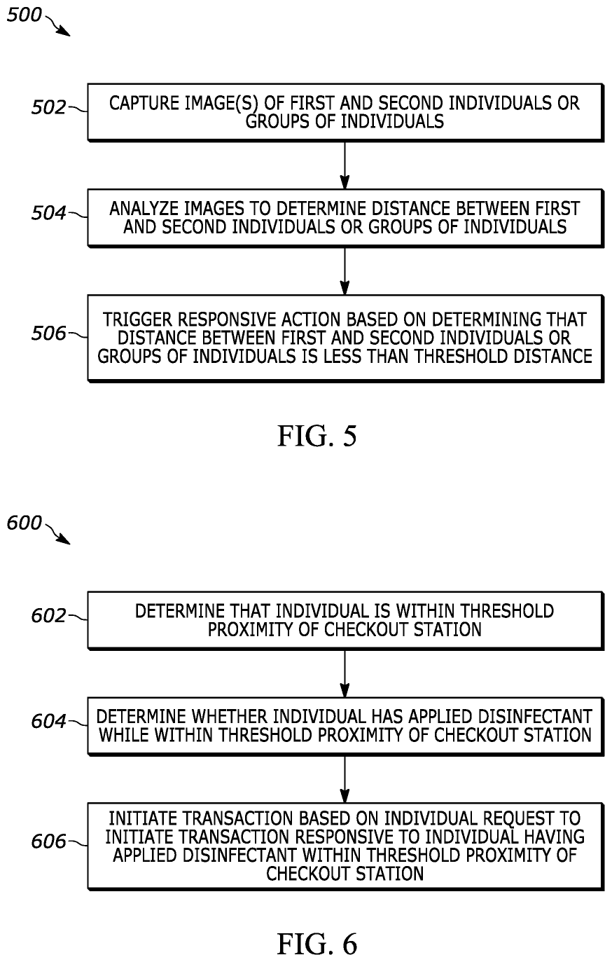

FIG. 5 illustrates a block diagram of an example process 500 as may be implemented by the system of FIG. 1, for implementing example methods and/or operations described herein, e.g., including social distancing verification methods discussed as being performed by the social distancing verification application 126 of the checkout workstation 104. At block 502, images of first and second individuals (or first and second groups of individuals) may be captured by an image sensor. At block 504, the images of the first and second individuals (or first and second groups of individuals) may be analyzed to determine distances between the first and second individual (or between each of the first group of individuals and the second groups of individuals). At block 506, a responsive action may be triggered based on determining that the distance between the first and second individual (or between any of the first group of individuals and any of the second group of individuals) is less than a threshold distance.

FIG. 6 illustrates a block diagram of an example process 600 as may be implemented by the system of FIG. 1, for implementing example methods and/or operations described herein, including disinfectant verification methods, e.g., as discussed as being performed by the disinfection verification application 128 of the checkout workstation 104. At block 602, a determination may be made that an individual is within a threshold proximity of a checkout workstation. At block 604, a determination may be made as to whether the individual has applied disinfectant (e.g., to his or her hands, or to the checkout workstation) while within the threshold proximity of the checkout workstation. At block 606, a transaction may be initiated based on a request from the individual within the threshold proximity of the checkout workstation responsive to determining that the individual has applied disinfectant while within the threshold proximity of the checkout workstation.

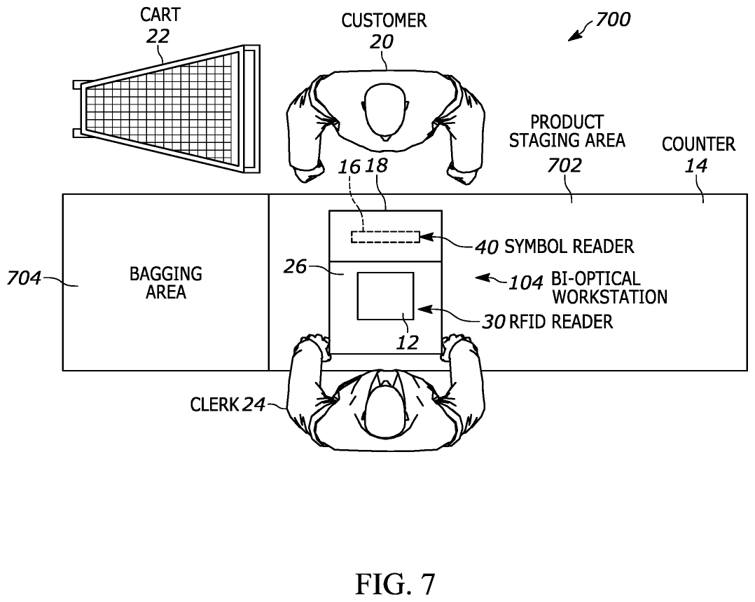

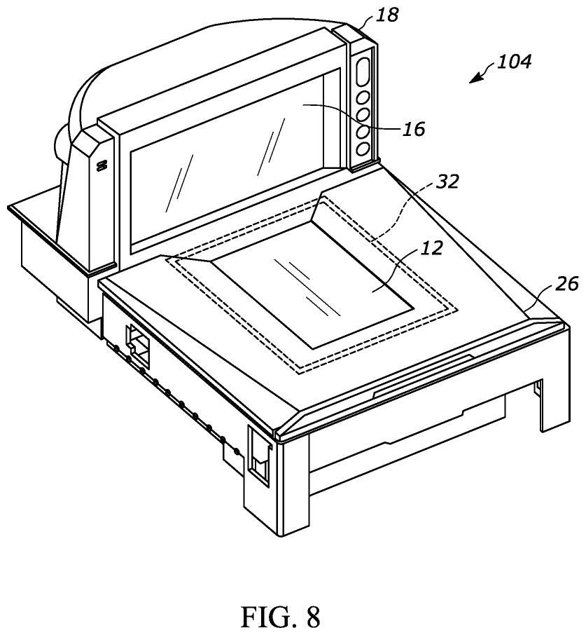

Turning now to FIG. 7, depicted therein is an example retail checkout system 700 that includes a dual window, multi-plane, bi-optical, point-of-transaction, retail checkout workstation 104 used by retailers at a retail checkout counter 14 in an aisle to process transactions involving the purchase of retail products associated with, or bearing, an identifying target, such as a barcode or other symbol. In a typical retail venue, a plurality of such workstations 104 are arranged in a plurality of checkout aisles. As best seen in FIG. 8, the workstation 104 has a generally horizontal, planar, generally rectangular, bed window 12 supported by a horizontal bed 26. The bed window 12 is either elevated, or set flush, with the counter 14. A vertical or generally vertical, i.e., slightly tilted, (referred to as “upright” hereinafter) planar, generally rectangular, tower window 16 is set flush with, or, as shown, recessed into, a raised tower 18 above the counter 14. The workstation 104 either rests directly on the counter 14, or preferably, rests in a cutout or well formed in the counter 14. Both the bed and tower windows 12, 16 are typically positioned to face and be accessible to a clerk 24 (FIG. 7) standing at one side of the counter 14 for enabling the clerk 24 to interact with the workstation 104. Alternatively, in a self-service checkout, the bed and tower windows 12, 16 are typically positioned to face and be accessible to a customer 20.

FIG. 7 also schematically depicts that a product staging area 702 is located on the counter 14 at one side of the workstation 104. The products are typically placed on the product staging area 702 by the customer 20 standing at the opposite side of the counter. The customer 20 typically retrieves the individual products for purchase from a shopping cart 22 or basket for placement on the product staging area 102. A non-illustrated conveyor belt could be employed for conveying the products to the clerk 24.

FIG. 7 schematically depicts that the workstation 104 has a bar code symbol reader 40, for example, a plurality of imaging readers, each including a solid-state imager for capturing light passing through either or both windows 12, 16 from a one- or two-dimensional symbol over an imaging field of view (FOV). In typical use, the clerk 24 may process each product bearing a UPC symbol thereon, past the windows 12, 16 by swiping the product across a respective window, or by presenting the product by holding it momentarily steady at the respective window, before passing the product to a bagging area 704 that is located at the opposite side of the workstation 104. The symbol may be located on any of the top, bottom, right, left, front and rear, sides of the product, and at least one, if not more, of the imagers will capture the return light returning from the symbol through one or both windows 12, 16 as an image.

In some examples, the workstation 104 may further include an RFID reader 30 that detects return RF energy returning from RFID tags associated with the products passing through the workstation 104 past either or both windows 12, 16. Although the workstation 104 has been illustrated as a dual-window workstation, it will be understood that the readers 30 and/or 40 could be installed in other types of workstations, for example, a flat bed scanner having a single horizontal window, or a vertical slot scanner having a single upright window. As previously mentioned, either or both windows 12, 16 is transmissive to light, for example, is constituted of glass or plastic. In the case of imaging readers, an illumination source emits illumination light in one direction through the windows 12, 16, and the return illumination light that is reflected and/or scattered from the symbol passes in the opposite direction to the imagers. In the case of moving laser beam readers, a laser emits laser light in one direction through the windows 12, 16, and the return laser light that is reflected and/or scattered from the symbol passes in the opposite direction to a photodetector. The bed 26 and the tower 18 of the workstation 104 together comprise a housing or chassis for supporting the windows 12, 16.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The above description refers to a block diagram of the accompanying drawings. Alternative implementations of the example represented by the block diagram includes one or more additional or alternative elements, processes and/or devices. Additionally or alternatively, one or more of the example blocks of the diagram may be combined, divided, re-arranged or omitted. Components represented by the blocks of the diagram are implemented by hardware, software, firmware, and/or any combination of hardware, software and/or firmware. In some examples, at least one of the components represented by the blocks is implemented by a logic circuit. As used herein, the term “logic circuit” is expressly defined as a physical device including at least one hardware component configured (e.g., via operation in accordance with a predetermined configuration and/or via execution of stored machine-readable instructions) to control one or more machines and/or perform operations of one or more machines. Examples of a logic circuit include one or more processors, one or more coprocessors, one or more microprocessors, one or more controllers, one or more digital signal processors (DSPs), one or more application specific integrated circuits (ASICs), one or more field programmable gate arrays (FPGAs), one or more microcontroller units (MCUs), one or more hardware accelerators, one or more special-purpose computer chips, and one or more system-on-a-chip (SoC) devices. Some example logic circuits, such as ASICs or FPGAs, are specifically configured hardware for performing operations (e.g., one or more of the operations described herein and represented by the flowcharts of this disclosure, if such are present). Some example logic circuits are hardware that executes machine-readable instructions to perform operations (e.g., one or more of the operations described herein and represented by the flowcharts of this disclosure, if such are present). Some example logic circuits include a combination of specifically configured hardware and hardware that executes machine-readable instructions. The above description refers to various operations described herein and flowcharts that may be appended hereto to illustrate the flow of those operations. Any such flowcharts are representative of example methods disclosed herein. In some examples, the methods represented by the flowcharts implement the apparatus represented by the block diagrams. Alternative implementations of example methods disclosed herein may include additional or alternative operations. Further, operations of alternative implementations of the methods disclosed herein may combined, divided, re-arranged or omitted. In some examples, the operations described herein are implemented by machine-readable instructions (e.g., software and/or firmware) stored on a medium (e.g., a tangible machine-readable medium) for execution by one or more logic circuits (e.g., processor(s)). In some examples, the operations described herein are implemented by one or more configurations of one or more specifically designed logic circuits (e.g., ASIC(s)). In some examples the operations described herein are implemented by a combination of specifically designed logic circuit(s) and machine-readable instructions stored on a medium (e.g., a tangible machine-readable medium) for execution by logic circuit(s).

As used herein, each of the terms “tangible machine-readable medium,” “non-transitory machine-readable medium” and “machine-readable storage device” is expressly defined as a storage medium (e.g., a platter of a hard disk drive, a digital versatile disc, a compact disc, flash memory, read-only memory, random-access memory, etc.) on which machine-readable instructions (e.g., program code in the form of, for example, software and/or firmware) are stored for any suitable duration of time (e.g., permanently, for an extended period of time (e.g., while a program associated with the machine-readable instructions is executing), and/or a short period of time (e.g., while the machine-readable instructions are cached and/or during a buffering process)). Further, as used herein, each of the terms “tangible machine-readable medium,” “non-transitory machine-readable medium” and “machine-readable storage device” is expressly defined to exclude propagating signals. That is, as used in any claim of this patent, none of the terms “tangible machine-readable medium,” “non-transitory machine-readable medium,” and “machine-readable storage device” can be read to be implemented by a propagating signal.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

1. A method, comprising:
capturing, by an image sensor associated with a checkout workstation, one or more images of an individual within a threshold proximity of the checkout workstation;
analyzing, by one or more processors, the one or more images to determine whether the individual is wearing personal protective equipment (PPE); and
triggering, by the one or more processors, one or more actions based on determining that the individual is not wearing PPE.

2. The method of claim 1, further comprising:
determining, by one or more processors, an indication that the individual has left the checkout workstation, based on of one or more of: (i) determining that the individual has completed a transaction at the checkout workstation; (ii) determining that no transaction activity has occurred at the checkout workstation for greater than a threshold period of time; or (iii) failing to capture, by the image sensor associated with the checkout workstation, images of the individual within the threshold proximity of the checkout workstation.

3. The method of claim 2, wherein triggering the one or more actions based on determining that the individual is not wearing PPE includes:
triggering, by the one or more processors, an alert for an employee associated with the checkout workstation indicating that the checkout workstation should be disinfected after determining the indication that the individual has left the checkout workstation.

4. The method of claim 1, wherein triggering the one or more actions based on determining that the individual is not wearing PPE includes:
triggering, by the one or more processors, an alert for an employee associated with the checkout workstation indicating that the employee should bring PPE to the individual.

5. The method of claim 2, wherein triggering the one or more actions based on determining that the individual is not wearing PPE includes:
initiating, by the one or more processors, an automatic disinfection of the checkout workstation after determining the indication that the individual has left the checkout workstation.

6. The method of claim 5, wherein the automatic disinfection includes activating one or more of: a UV disinfection light, a disinfection aerosol, a disinfection wipe, or a robotic cleaning machine dispatched to the checkout workstation.

7. The method of claim 2, wherein triggering the one or more actions based on determining that the individual is not wearing PPE includes:
triggering, by the one or more processors, an alert for an employee associated with the checkout workstation indicating that the checkout workstation should be closed or locked to other individuals after determining the indication that the individual has left the checkout workstation.

8. The method of claim 2, wherein triggering the one or more actions based on determining that the individual is not wearing PPE includes:
causing, by the one or more processors, the checkout workstation to be automatically closed or locked to other individuals after determining the indication that the individual has left the checkout workstation.

9. The method of claim 8, wherein causing the checkout workstation to be automatically closed or locked includes activating or deactivating an indicator light associated with the checkout workstation.

10. The method of claim 1, wherein triggering the one or more actions based on determining that the individual is not wearing PPE includes:
generating, by the one or more processors, an alert to be displayed to the individual indicating that the individual must don PPE.

11. The method of claim 1, wherein triggering the one or more actions based on determining that the individual is not wearing PPE includes:
analyzing, by the one or more processors, the one or more images of the individual in order to identify the individual.

12. The method of claim 11, further comprising:
generating, by the one or more processors, an alert for an employee associated with the checkout workstation including an indication of the identified individual.

13. The method of claim 11, wherein the one or more images of the identified individual within the threshold proximity of the checkout workstation are captured at a first time, the method further comprising:
capturing, at a second time after the individual has left the checkout workstation, by an image sensor associated with the checkout workstation, one or more images of the identified individual within the threshold proximity of the checkout workstation;
analyzing, by one or more processors, the one or more images captured at the second time to determine whether the identified individual is wearing PPE; and
preventing, by the one or more processors, based on determining that the identified individual is not wearing PPE at the second time, the checkout workstation to be automatically closed or locked to the identified individual.

14. The method of claim 1, wherein triggering the one or more actions based on determining that the individual is not wearing PPE includes:
causing, by the one or more processors, the checkout workstation to one or more of: (i) prevent a transaction from being initiated by the individual, or (ii) cease a pending transaction that has been initiated by the individual.

15. The method of claim 1, wherein triggering the one or more actions based on determining that the individual is not wearing PPE includes:
causing, by the one or more processors, the checkout workstation or a dispensing device associated with the checkout workstation to automatically dispense PPE for the individual.

16. The method of claim 1, wherein triggering the one or more actions based on determining that the individual is not wearing PPE includes:
causing, by the one or more processors, the checkout workstation or a dispensing device associated with the checkout workstation to automatically dispense a stylus for operating the checkout workstation.

17. The method of claim 1, wherein analyzing the one or more images to determine whether the individual is wearing PPE further includes determining whether the individual is wearing PPE correctly; and wherein triggering the one or more actions is based on determining that the individual is not wearing PPE correctly.

18. The method of claim 17, wherein triggering the one or more actions based on determining that the individual is not wearing PPE correctly includes:
generating, by the one or more processors, an alert to be displayed to the individual indicating instructions for wearing PPE correctly.

19. The method of claim 18, wherein determining that the individual is not wearing PPE correctly includes determining an indication of a specific error in the way that the individual is wearing PPE, and wherein the alert indicates instructions for correcting the specific error.

20. The method of claim 1, wherein the individual is a customer.

21. The method of claim 1, wherein the individual is an employee associated with the checkout workstation.

22. The method of claim 1, wherein determining whether the individual is wearing PPE further includes determining whether the individual is wearing one or more particular types of PPE.

23. The method of claim 22, wherein the one or more particular types of PPE include one or more of: a mask, a face shield, goggles, gloves, a respirator, a hairnet or other head covering, or an isolation gown.

24. The method of claim 1, wherein triggering the one or more actions is further based on determining that a number of individuals, that have been within the threshold proximity of the checkout workstation without wearing PPE, within a particular timeframe, is greater than a threshold number.

25. The method of claim 24, wherein the particular timeframe begins when the checkout workstation is disinfected.

26. The method of claim 1, wherein triggering the one or more actions is further based on determining that a collective total amount of time, during which any individuals that have been within the threshold proximity of the checkout workstation without wearing PPE, is greater than a threshold amount of time.

27. The method of claim 1, wherein analyzing the one or more images to determine whether the individual is wearing PPE includes one or more of:
(i) analyzing the one or more images to determine whether the mouth of the individual appears in the one or more images;
(ii) analyzing the one or more images to determine whether a demarcation or a color transition below the eyes of the individual, indicative of a face covering, appears in the one or more images;
(iii) analyzing the one or more images to determine whether strings or loops over the ears of the individual appear in the one or more images;
(iv) analyzing the one or more images to determine whether nails or cuticles of the individual appear in the one or more images; or
(v) analyzing the one or more images to determine whether wrinkles of hands of the individual appear in the one or more images.

28. The method of claim 1, further comprising:
analyzing, by one or more processors, the one or more images to determine whether the individual has coughed, sneezed, or spoken while within the threshold proximity of the checkout workstation; and
wherein triggering the one or more actions is further based on determining that the individual has coughed, sneezed, or spoken while within the threshold proximity of the checkout workstation.

29. The method of claim 1, wherein the image sensor is a depth image sensor.

30. The method of claim 1, further comprising:
detecting, by a thermal image sensor, the individual within the threshold proximity of the checkout workstation.

31. The method of claim 30, further comprising:
detecting, by the thermal image sensor, a body temperature associated with the individual;
and wherein triggering the one or more actions is further based on the body temperature associated with the individual being greater than a threshold body temperature.

32. The method of claim 30, wherein the thermal image sensor is an infrared image sensor.

33-43. (canceled)
